(19) United States
(12) Patent Application Publication
(10) Pub. No.: US 2021/0177656 A1
(43) Pub. Date: Jun. 17, 2021

(54) OCULAR TISSUE PERFORATION DEVICE

(71) Applicant: GLAUKOS CORPORATION, San Clemente, CA (US)

(72) Inventors: Mark Gallardo, San Clemente, CA (US); David S. Haffner, Mission Viejo, CA (US); Gary Rangel-Friedman, San Clemente, CA (US)

(21) Appl. No.: 17/125,466

(22) Filed: Dec. 17, 2020

Related U.S. Application Data
(60) Provisional application No. 62/949,267, filed on Dec. 17, 2019.

(51) Int. Cl. A61F 9/007

(52) U.S. Cl. CPC A61F 9/00754; A61F 9/00781

(57) ABSTRACT

An instrument to perforate ocular tissue can include a handle configured to be grasped by a user, the handle including a proximal handle end and a distal handle end and a dissection cannula configured for at least partial insertion into a patient eye, wherein the dissection cannula can include a proximal cannula end configured to interface with the handle, a distal cannula or trephine end including an opening into a cannula bore, and an indicator spaced from the distal cannula end at a spacing to indicate or control insertion depth of the distal cannula end into the eye.

### PRIORITY CLAIM AND CROSS-REFERENCE TO RELATED APPLICATION

This application claims priority to and benefit of U.S. Provisional Application No. 62/949,267, filed Dec. 17, 2019, entitled “OCULAR TISSUE PERFORATION DEVICE”, the entire contents of which are hereby incorporated in its entirety including all tables, figures, and claims.

### TECHNICAL FIELD

This document pertains generally, but not by way of limitation, to surgery of the eye.

### BACKGROUND

Glaucoma is a progressive disease of the eye with many risk factors the sole modifiable risk factor being elevated intraocular pressure or IOP. Elevated IOP is often associated with increased resistance to flow throughout the conventional outflow system with a majority of resistance residing in the trabecular meshwork and inner wall of Schlemm's canal, a porous tissue complex, separating the anterior chamber (fluid filled intraocular space) from Schlemm's canal and the distal collector system. Surgical techniques to improve drainage of aqueous fluid from the eye, such as goniotomy, trabeculotomy, and trabecular micro-bypass with micro-stents can be prohibitively expensive.

Haffner U.S. Pat. No. 9,597,230 mentions intraocular implants and delivery instruments for treating ophthalmic conditions and ocular disorders.

Berlin U.S. Pat. No. 10,390,993 mentions an apparatus having an inserter device with a shaft disposed in an interior space and an intraocular implant.

Vandiest U.S. Patent Publication No. 2019/0038462 mentions an ocular implant system in which an implant is deployed into a posterior space of the eye for reducing intraocular pressure in the eye.

### SUMMARY

The present inventors have recognized, among other things, that there is a need in the art for systems and methods that can perforate and remove intraocular tissue, such as to create a passage of lesser resistance between the anterior chamber and Schlemm's canal. The passage can increase the flow of aqueous humor from the anterior chamber and into Schlemm's canal and distal collector, thereby reducing the IOP in the patient eye.

An instrument to perforate ocular tissue can include a handle configured to be grasped by a user, the handle including a proximal handle end and a distal handle end and a dissection cannula or trephine configured for at least partial insertion into a patient eye, wherein the dissection cannula can include a proximal end configured to interface with the handle, a distal end including an opening into a trephine bore, and an indicator spaced from the distal cannula end at a spacing to indicate or control insertion depth of the distal cannula end into the eye.

In light of the disclosure herein, and without limiting the scope of the invention in any way, in a first aspect of the present disclosure, which may be combined with any other aspect listed herein unless specified otherwise, an instrument to perforate ocular tissue includes a handle and a dissection cannula. The handle is configured to be grasped by a user. The handle includes a proximal handle end and a distal handle end. The dissection cannula is configured for at least partial insertion into a patient eye. The dissection cannula includes a proximal shaft configured to interface with the handle and a distal cannula end. The distal cannula end includes an opening into a cannula bore. The dissection cannula further includes an indicator spaced from the distal cannula end at a spacing to indicate or control insertion depth of the distal cannula end into the eye.

In a second aspect of the present disclosure, which may be combined with any other aspect listed herein unless specified otherwise, the distal cannula end includes a beveled edge or trephine to penetrate ocular tissue.

In a third aspect of the present disclosure, which may be combined with any other aspect listed herein unless specified otherwise, the beveled edge includes at least one of a single bevel or a double bevel.

In a fourth aspect of the present disclosure, which may be combined with any other aspect listed herein unless specified otherwise, the beveled edge is sloped toward an interior surface of the cannula.

In a fifth aspect of the present disclosure, which may be combined with any other aspect listed herein unless specified otherwise, the instrument includes a serrated edge extending about at least a portion of the distal cannula end.

In a sixth aspect of the present disclosure, which may be combined with any other aspect listed herein unless specified otherwise, the serrated edge extends about the entire distal cannula end.

In a seventh aspect of the present disclosure, which may be combined with any other aspect listed herein unless specified otherwise, the indicator includes a backstop extending laterally wider than the distal cannula end.

In an eighth aspect of the present disclosure, which may be combined with any other aspect listed herein unless specified otherwise, the backstop includes a collar extending about at least a portion of the exterior surface of the cannula.

In a ninth aspect of the present disclosure, which may be combined with any other aspect listed herein unless specified otherwise, the indicator includes an elastic band configured to be user-adjustable in location on the cannula to define the spacing.

In a tenth aspect of the present disclosure, which may be combined with any other aspect listed herein unless specified otherwise, the instrument includes a pressure source in communication with the cannula bore to adjust pressure in the cannula bore.

In an eleventh aspect of the present disclosure, which may be combined with any other aspect listed herein unless specified otherwise, the pressure source is configured to generate enough negative pressure in the cannula bore to draw ocular tissue into the bore.

In a twelfth aspect of the present disclosure, which may be combined with any other aspect listed herein unless specified otherwise, the pressure source is configured to generate enough negative pressure in the cannula bore to cut the ocular tissue against the distal cannula end.

In a thirteenth aspect of the present disclosure, which may be combined with any other aspect listed herein unless specified otherwise, the handle is integral with the dissection cannula.

In a fourteenth aspect of the present disclosure, which may be combined with any other aspect listed herein unless specified otherwise, a method of using an instrument includes inserting a distal cannula end of the instrument into a patient eye to locate the distal cannula end against intraocular tissue, and applying force between the instrument and the eye to perforate the intraocular tissue and capture the perforated intraocular tissue for removal from the eye.

In a fifteenth aspect of the present disclosure, which may be combined with any other aspect listed herein unless specified otherwise, inserting includes locating the instrument against a trabecular meshwork of the eye.

In a sixteenth aspect of the present disclosure, which may be combined with any other aspect listed herein unless specified otherwise, applying force includes applying at least one of a linear force or a rotary force to the instrument located against the ocular tissue to perforate the tissue.

In a seventeenth aspect of the present disclosure, which may be combined with any other aspect listed herein unless specified otherwise, applying force includes applying linear force.

In an eighteenth aspect of the present disclosure, which may be combined with any other aspect listed herein unless specified otherwise, applying force includes applying rotary force.

In a nineteenth aspect of the present disclosure, which may be combined with any other aspect listed herein unless specified otherwise, applying force includes generating negative pressure in a bore of the instrument.

In a twentieth aspect of the present disclosure, which may be combined with any other aspect listed herein unless specified otherwise, the method includes indicating or controlling penetration depth of the distal cannula end into the ocular tissue.

This summary is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows a side view of an example instrument 100.

FIG. 2A shows a side view of an example distal cannula/trephine end.

FIG. 2B shows an end view of the example distal cannula/trephine end of FIG. 2A.

FIG. 3A shows a cross sectional view of an example distal cannula/trephine end.

FIG. 3B shows an end view of the example distal cannula/trephine end of FIG. 3A.

FIG. 4A shows a cross sectional view of an example distal cannula/trephine end.

FIG. 4B shows an end view of the example distal cannula/trephine end of FIG. 4A.

FIG. 5 shows an embodiment of an example instrument.

FIG. 6 shows a block diagram of an example method of using the instrument.

FIGS. 7 to 10 show an example instrument utilizing a hypodermic needle housing, with a trephine disposed therein.

FIG. 11 shows a dissection trephine, inserted into the ocular environment.

FIG. 12 shows a dissection trephine, with a support collar.

FIG. 13 shows alternative geometries of a support collar.

FIG. 14 shows an alternative instrument, with a control wheel.

FIG. 15 shows an alternative instrument, with a thumbwheel.

### DETAILED DESCRIPTION

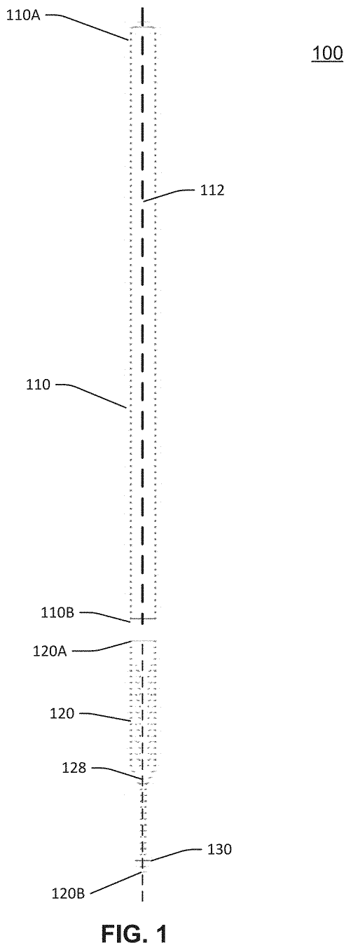

FIG. 1 shows a side view of an example instrument 100, such as for performing surgery on a patient eye including goniotomy. The instrument can include a handle 110 with a proximal handle end 110A and a distal handle end 110B, and a dissection trephine 120 with a proximal shaft end 120A and a distal trephine end 120B. In an example, the distal handle end 110B can interface to the proximal shaft end 120A, such as to form the instrument 100.

The instrument 100, such as the distal cannula/trephine end 120B, can be inserted into the anterior chamber of the patient eye, such as through an incision in the cornea of the eye. Once inserted, the distal cannula end 120B can be located proximally to intraocular tissue, such as to contact the intraocular tissue. The distal cannula end 120B can include a cutting surface, such as to cut, core or incise the intraocular tissue. In an example, the cutting surface can include a cutting trephine.

The handle 110 can include an elongated member, such as to be grasped by a user for insertion of at least part of the instrument 100 into the intraocular space. The handle 110 can be defined by a major handle axis 112. The major handle axis 112 can extend through at least a portion of the handle 110, such as through a centroid at any given cross section of the handle 110. The cross section of the handle 110 can assume a generally symmetrical shape, including a bilaterally symmetric shape such as a circular or rectangular shape, or a generally non-symmetrical shape. The cross section of the handle 110 can change along the length of the major handle axis 112, such as to enhance ergonomic functionality of the instrument 100. In an example, the handle 110 can include a generally rectangular cross section near the proximal handle end 110A and transition to a generally circular cross section near the distal handle end 110B, such as to allow a user to more easily grasp the instrument 100. The handle 110 can include a handle bore, such as a channel extending along the major handle axis 112 through at least a portion of the handle 110. In an example, the handle bore can extend completely through the handle 110, such as along the major handle axis 112 from the proximal handle end 110A to the distal handle end 110B.

The dissection trephine 120 can include an elongated member, such as defined by a major cannula axis 128 and an exterior cannula surface 132, such as the surface of the dissection trephine 120 facing radially outward with respect to the major cannula axis 128. The major cannula axis 128 can extend through at least a portion of the dissection trephine 120, such as through a centroid at any given cross section of the dissection trephine 120. The cross section of the dissection trephine 120 can assume a generally symmetrical shape, including a bilaterally symmetric shape such as a circular or rectangular shape, or a generally non-symmetrical shape. The cross section of the dissection trephine 120 can change along the length of the major cannula axis 128, such as to enhance functionality of the instrument 100. In an example, the dissection trephine 120 can include a generally circular cross section near the proximal shaft end 120A, such as to interface with the distal handle end 110B, and transition to a generally rectangular cross section near the distal cannula end 110B, such as to allow a user to create a rectangular opening in the intraocular tissue.

The dissection trephine 120 can include a cannula bore, such as a channel extending along the major cannula axis 128 through at least a portion of the dissection trephine 120. The cannula bore can be defined by an interior cannula surface 134, such as the surface of the cannula bore facing radially inward with respect to the major cannula axis 128. In an example, the cannula bore can extend completely through the cannula 110, such as along the major cannula axis 112 from the proximal shaft end 120A to the distal cannula end 120B. The cannula bore can communicate with the handle bore, such as when the distal handle end 110B interfaces to the proximal shaft end 120A, to form an instrument bore, such as a contiguous bore through the instrument 100. In an example, the instrument bore can include a continuous bore from the proximal handle end 110A to the distal cannula end 120B.

The dissection trephine 120 can vary in diameter, such as along the major cannula axis 128. In an example, the dissection trephine 120 can include a proximal cannula diameter, such as the diameter of the dissection trephine 120 at the proximal shaft end 120A, and a distal cannula diameter, such as the diameter of the dissection trephine 120 at the distal cannula end 120B. The shape of the proximal shaft end 120A or the distal cannula end 120B can assume any geometrical shape, such as a symmetric shape including at least one of a circular, rectangular, triangular, or polygonal shape, or a non-symmetric shape. In an example, the diameter of the distal cannula end 120B can include a circular cross-section with an outer diameter, such as an outer diameter in a range of about 100 micrometers to about 200 micrometers, inclusive.

The proximal shaft end 120A can be configured to form an interface with the distal handle end 110B, such as to removably attach the dissection trephine 120 to the handle 110 to form the instrument 100. The interface can include a friction interface, such as a friction interface formed by locating the distal handle end 110A with respect to the proximal shaft end 120A, such as by inserting the distal handle end 120A into the proximal shaft end 120A. The interface can include a threaded interface.

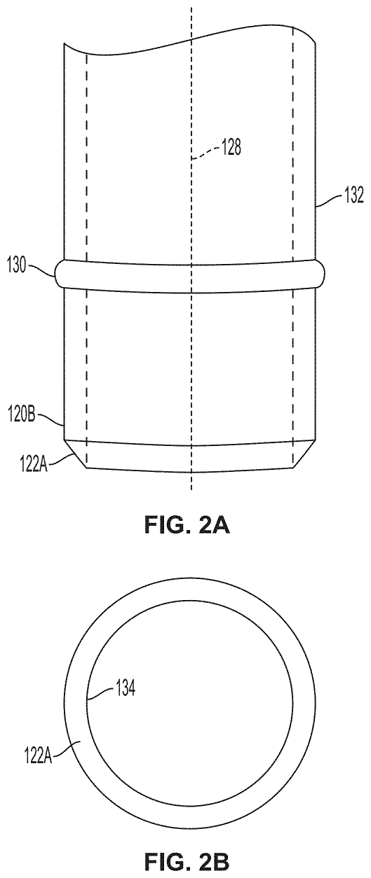

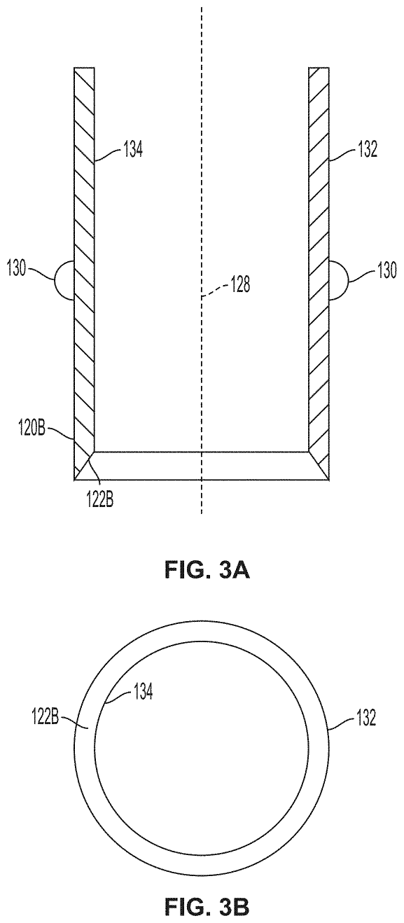

FIG. 2A shows a side view of an example distal cannula end 120B and FIG. 2B shows an end view of the example distal cannula end 120B. The distal cannula end 120B can be configured to incise intraocular tissue of the patient eye, such as with a cutting edge 122 formed in the distal cannula end 120B.

The cutting edge 122 can include a beveled edge, such as by at least one of a single bevel cutting edge or a double bevel cutting edge. The beveled edge can extend about at least a portion of the distal cannula end 120B, such as about 10%, 20%, 30%, 40%, 50%, 60%, 70%, 80%, 90%, or about 100% of the distal cannula end 120B. In an example, the beveled edge can extend about the entire distal cannula end 120B, such as to form a continuous bevel around the distal cannula end 120B.

The cutting edge 122 can include an exterior bevel cutting edge 122A, such as a cutting edge 122 beveled toward the exterior cannula surface 132 as shown in FIG. 2A. The exterior bevel cutting edge 122A can be configured to cut an intraocular tissue plug, such as a portion of intraocular tissue, with a diameter that can be approximately equal to or less than the diameter of the cannula bore, such as the inner diameter of the distal cannula end 120B. In an example, the tissue plug can move with respect to the interior cannula surface 134, such as slide freely with respect to the interior cannula surface 134. The tissue plug can subsequently be removed from the intraocular space, such as by drawing the tissue plug through the instrument bore, such as at least one of the cannula bore or the handle bore. The tissue plug can be drawn from the intraocular space by creating a differential pressure in the instrument bore, such as with a pressure source 140 in fluidic communication with the instrument bore, the pressure source 140 including a suction source. In an example, the suction source can include the pressure source 140 configured to create a negative pressure level environment relative to ambient atmospheric pressure in the instrument bore.

The cutting edge 122 can include a serrated edge, such as similar to a saw blade. The serrated edge can extend about at least a portion of the distal cannula end 120B such as about 10%, 20%, 30%, 40%, 50%, 60%, 70%, 80%, 90%, or about 100% of the distal cannula end 120B. In an example, the serrated edge can extend about the entire distal cannula end 120B.

The instrument can include a depth indicator such as an element configured to indicate depth of penetration of the distal cannula end 120B into the intraocular tissue. The depth indicator can be located in proximity to the distal cannula end 120B, such as a known distance from the distal cannula end 120B. The known distance can include a distance range, such as at least one of a distance range of about 50 micrometers to about 250 micrometers from the distal cannula end 120B or a distance range of about 100 micrometers to about 200 micrometers from the distal cannula end 120B.

The depth indicator can include an indicia mark, such as at least one of a printed indicia mark or an etched indicia mark indicating a known distance from the distal cannula end 120B. In an example, the indicia mark can be attached to the exterior cannula surface 132 to serve as an indication of distance from the distal cannula end 120B to the indicia mark. The indicia mark can include one or more indicia marks, such as one or more indicia marks indicating regular intervals in a distance range. In an example, the indicia mark can include one or more indicia marks denoting a distance range of about 50 micrometers to about 200 micrometers from the distal cannula end 120B, such as by locating an indicia mark at each of 50, 100, 125, 150, 175, and 200 micrometers from the distal cannula end 120B.

The depth indicator can include a backstop 130, such as an element configured to limit the insertion depth of the distal cannula end 120B into the ocular tissue. The backstop 130 can limit insertion depth, such as by locating the backstop 130 to interfere with a surface of the ocular tissue after insertion of the distal cannula end 120B into the ocular tissue. In an example, the backstop can be configured to limit insertion depth of the distal cannula end 120B into the trabecular meshwork of a patient eye, such as to prevent damage to Schlemm's canal and sclera during excision of the tissue plug. In an example, the backstop 130 can include at least one of a band or a collar, such as made from an elastic material, that can stretch about the exterior cannula surface 132, such as to secure the band or collar to the exterior cannula surface 132 by friction, and can be located on the exterior cannula surface 132 at distance from the distal cannula end 120B specified by a user, such as to allow the user to set the insertion depth of the distal cannula end 120B.

The backstop 130 can be located on a surface of the dissecting cannula 120, such as on at least one of the exterior cannula surface 132 or the interior cannula surface 134. The backstop 130 can extend about at least a portion of the dissecting cannula 120, such as the backstop 130 can extend about 10%, 20%, 30%, 40%, 50%, 60%, 70%, 80%, 90%, or about 100% of at least one of the periphery of the internal cannula surface 134 or the periphery of the external cannula surface 132. In an example, the backstop 130 can form a contiguous structure about at least one of the periphery of the exterior cannula surface 132 or the periphery of the interior cannula surface 134.

The backstop 130 can extend radially from a surface of the dissecting cannula 120, such as in any direction perpendicular to the major cannula axis 128. In an example, the backstop 130 can extend radially outward from the exterior cannula surface 132, such as the inner diameter of the backstop 130 can be equal to the outer diameter of the dissection trephine 120 and the outer diameter of the backstop 130 can be greater than the diameter of the dissection trephine 120. In an example, the backstop 130 can extend radially inward from the interior cannula surface 134 toward the major cannula axis 128, such as the outer diameter of the backstop 130 can be equal to the inner diameter of the dissection trephine 120 and the inner diameter of the backstop 130 can be less than the diameter of the dissection trephine 120.

The cross-section of the backstop 130, such as a cross-section located in a plane extending radially from the major cannula axis 128 and parallel to the major cannula axis 128, can assume any geometrical shape, such as a symmetric shape including at least one of a circular, rectangular, triangular, or polygonal shape, or a non-symmetric shape.

The backstop 130 can be positioned with reference to the major cannula axis 128, such as a plane parallel to a perimeter of the backstop 130 can form a backstop angle with respect to the major cannula axis 128. The backstop angle can be established, such as to control ocular tissue perforation depth during surgery using a non-perpendicular approach to the ocular tissue. The backstop angle can include an angle of about 45 degrees, 50 degrees, 55 degrees, 60 degrees, 65 degrees, 70 degrees, 75 degrees, 80 degrees, 85 degrees, or about 90 degrees with respect to the major cannula axis 128. In an example, the backstop 130 can be located in proximity to the distal cannula end 120B and perpendicular to the major cannula axis 128.

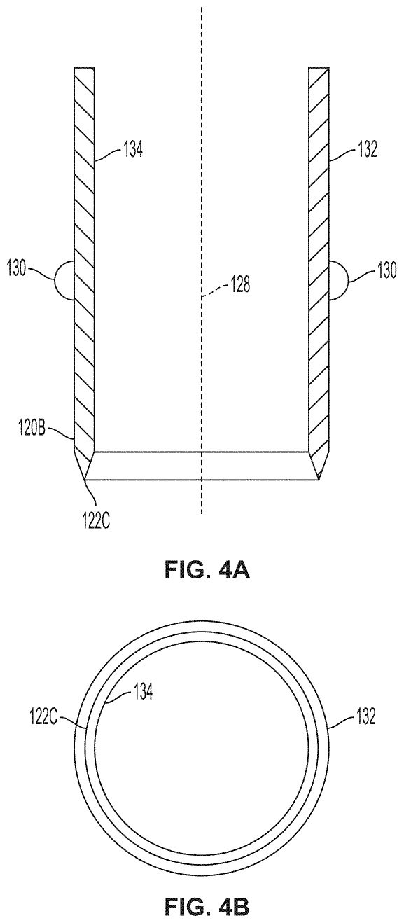

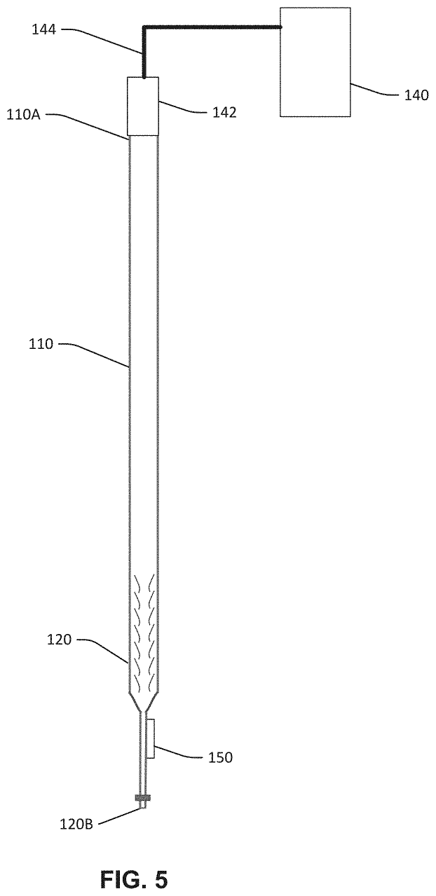

FIG. 3A shows a cross sectional view of an example distal cannula end 120B and FIG. 3B shows an end view of the example distal cannula end 120B. The cutting edge 122B can include an interior bevel cutting edge 122B, such as a cutting edge 122 beveled toward the interior cannula surface 134 as shown in FIGS. 3A and 3B. The interior bevel cutting edge 122B can be configured to cut an intraocular tissue plug, such as a portion of intraocular tissue, with a diameter that can be approximately equal to or greater than the diameter of the cannula bore, such as the diameter of the distal cannula end 120B. In an example, the diameter of the tissue plug can interfere with the diameter of the cannula bore, such as to create resistance to motion of the tissue plug with respect to the interior cannula surface 134. The tissue plug can subsequently be removed from the intraocular space, such as by withdrawing the instrument 100 from the intraocular space and expelling the tissue plug from the instrument bore. The tissue plug can be expelled from the instrument bore by creating a differential pressure in at least one of the cannula bore or the handle bore, such as with a pressure source in fluidic communication with at least one of the handle bore or the cannula bore and configured to create a positive pressure environment in at least one of the bores relative to ambient atmospheric pressure.

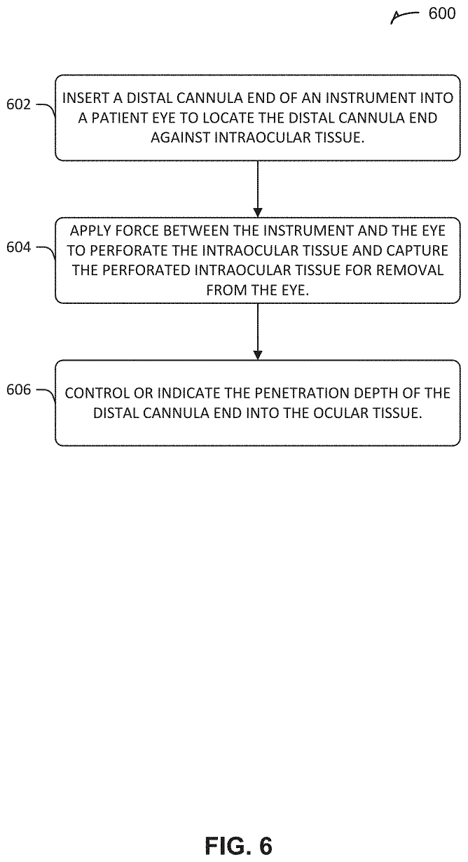

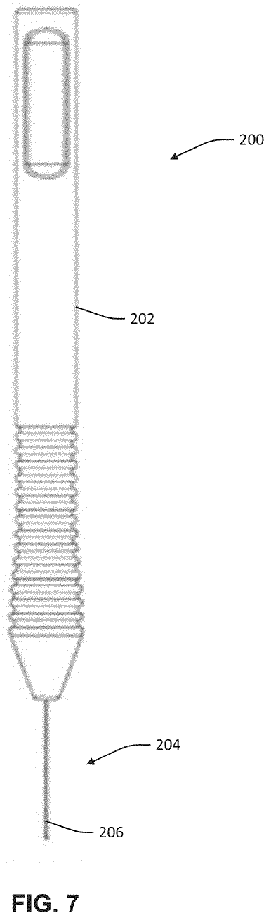

FIG. 4A shows a cross sectional view of an example distal cannula end 120B and FIG. 4B shows an end view of the example distal cannula end 120B. The cutting edge 122 can include a double bevel cutting edge 122C, such as a cutting edge 122 beveled toward the interior cannula surface 134 and the exterior cannula surface 132. The double bevel cutting edge 122C can be configured to cut an intraocular tissue plug, such as a portion of intraocular tissue, with a diameter that can be approximately equal to or greater than the diameter of the cannula bore, such as the diameter of the distal cannula end 120B. In an example, the diameter of the tissue plug can interfere with the diameter of the cannula bore, such as to create resistance to motion of the tissue plug with respect to the interior cannula surface 134. The tissue plug can subsequently be removed from the intraocular space, such as by withdrawing the instrument 100 from the intraocular space and expelling the tissue plug from the bore. The tissue plug can be expelled from the bore by creating a differential pressure in at least one of the cannula bore or the handle bore, such as with a pressure source in fluidic communication with at least one of the bores and configured to create a positive pressure environment in at least one of the bores relative to ambient atmospheric pressure.

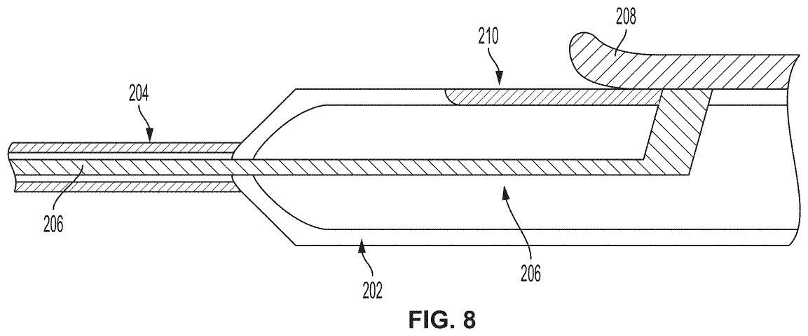

FIG. 5 shows an embodiment of an example instrument 100. The instrument 100 can include a pressure source 140, such as a pressure source capable of generating a differential pressure with respect to ambient pressure including atmospheric pressure. The pressure source 140 can generate at least one of a positive pressure level, such as a pressure level greater than ambient pressure, or a negative pressure level, such as a pressure level less than ambient pressure. The pressure source 140 can include an electrically-actuated source of pressure, such as a pump, or a manually-actuated source of pressure, such as a bellows or a syringe. The pressure source 140 can connect to the instrument 100, such as to vary pressure in the instrument bore, such as at least one of the handle bore or the cannula bore.

The pressure source 140 can connect to the instrument 100 at an interface 142, such as a luer-type fitting or a threaded connection for ease of connection with the pressure source 140. In an example, the pressure source 140 can connect to the instrument 100 at the interface 142 located at the proximal handle end 110A, such as to generate a negative pressure level in the instrument bore. In an example, the negative pressure level can be selected to draw tissue toward contact with the distal cannula end 120B, such as to cause intraocular tissue in proximity to the distal cannula end 120B to be drawn into the distal cannula end 120B.

The interface 142 can include a valve, such as a valve configured to limit the pressure level applied to the instrument bore. In an example, the interface 142 can be in fluidic communication with the pressure source 140, such as with a tube 144. The valve can include a check valve such as a check valve with at least one of a fixed or an adjustable cracking pressure. A cracking pressure can describe the pressure level condition, such as the differential pressure between the instrument bore and ambient pressure, under which the check valve can open. For example, the check valve can open when the differential pressure is greater than the cracking pressure and close when the differential pressure is less than the cracking pressure. In an example, pressure in the instrument bore can be limited to a safe negative pressure level, such as a negative pressure level that cannot cause damage to the intraocular tissue, with the use of the check valve and an appropriately specified cracking pressure.

Additionally or alternatively, instrument 100 can be connected to an irrigation/aspiration unit. In this particular embodiment, instrument 100 may be configured to aspirate trabecular meshwork.

The instrument 100 can include a sensor 150, such as a visualization sensor configured to visualize an intraocular surgical field. The visualization sensor can include a fiber optic sensor in proximity to the distal cannula end 120B, such as to monitor at least one of penetration of intraocular tissue through observation of the indicia mark or contact of the backstop 130 with the intraocular tissue. In an example, the fiber optic sensor can be located in proximity to the backstop 130, such as at the same distance from the distal cannula end 120B as the backstop 130.

The instrument 100 can include a sensor 150, such as a position sensor configured to sense a distance from the sensor 150 to the intraocular tissue. The position sensor can include a distance sensor, such as an ultrasonic distance sensor, to monitor distance of the backstop 130 with the intraocular tissue. In an example, the distance sensor can be located in proximity to the backstop 130, such as the same distance from the distal cannula end 120B at the backstop 130.

The sensor 150 can include a force sensor, such as a sensor 150 configured to sense the applied force between the instrument 100 and the intraocular tissue to be perforated. The sensor 150 can include a torque sensor, such as a sensor 150 configure to sense the applied torque between the instrument 100 and the intraocular tissue to be perforated.

The instrument 100 can include a handle 110 that can be integral with the dissecting cannula 120, such as the handle 110 and the dissecting cannula 120 can be permanently attached to form a unitary instrument. The unitary instrument can be formed from a single material, such as a metallic material that can be formed into the unitary instrument by a metal working process, such as at least one of a rolling or a drawing process. The unitary instrument can be formed from a polymer material, such as through a molding process including at least one of a spin molding process or an injection molding process.

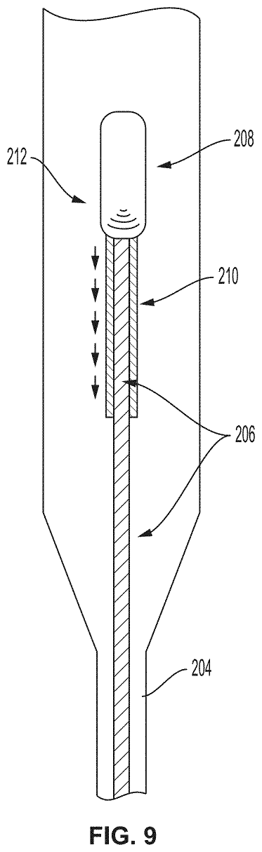

FIG. 6 shows a block diagram of an example method 600 of using the instrument 100, such as to perform a surgery on a patient eye. In an example, surgery of the patient eye can include a procedure to perforate a portion of the patient eye, such as a portion of intraocular tissue in the patient eye including at least one of a trabecular meshwork or an inner wall of Schlemm's canal in the eye. Perforation of the trabecular meshwork (TM) and Schlemm's canal inner wall (SCIW) can include the removal of TM/SCIW tissue from the intraocular space, such as to create a void in the TM/SCIW to allow aqueous humor in the anterior chamber of the patient eye to drain more readily into Schlemm's canal, such as to relieve elevated levels of intraocular pressure in the eye.

At 602, the distal cannula end 120B of the instrument 100 can be inserted into the patient eye, such as through an incision in the patient eye. The instrument 100 can be guided to locate the instrument 100, such as the distal cannula end 120B, against intraocular tissue. Intraocular tissue can include any tissue inside the eye including the anterior portion of the eye, such as at least one of the ciliary body, the suspensory ligament, or the ocular lens. In an example, the intraocular tissue can include the trabecular meshwork of the patient eye.

At 604, force can be applied between the instrument 100 and the eye, such as to perforate the intraocular tissue and capture the perforated intraocular tissue for removal from the eye. In an example, the perforated intraocular tissue can include a tissue plug. Application of the force can include the application of a linear force, such as a force to cause the instrument 100 to advance into the intraocular tissue along a path parallel to the major cannula axis 128. In an example, the linear force can be measured with a sensor 150, such as a force sensor configured to sense force applied between the instrument 100 and the intraocular tissue. The linear force can cause a plunge cut, such as to separate a portion of intraocular tissue from the intraocular tissue matrix by pressing the cutting edge 122 perpendicularly against the tissue. The portion of intraocular tissue separated from the intraocular tissue matrix, such as the tissue plug, can be contained within the instrument 100, such as within the cannula bore of the instrument 100.

Application of the force can include the application of a rotary force, such as a force to cause the instrument 100 to rotate about the major cannula axis 128. In an example, the rotary force can be measured with a sensor 150, such as a torque sensor configured to sense torque applied between the instrument 100 and the intraocular tissue. The rotary force can cause a slicing cut, such as to separate a portion of intraocular tissue from the intraocular tissue matrix by rotating, or otherwise sliding, the cutting edge 122 in contact with the intraocular tissue parallel to the intraocular tissue. The portion of intraocular tissue separated from the intraocular tissue matrix, such as the tissue plug, can be contained within the instrument 100, such as within the cannula bore of the instrument 100.

Application of the force can include the application of a rotary force in combination with a plunge force, such as to perforate the intraocular tissue with both a plunge cut and a slicing cut. The combination of rotary force and plunge force can improve perforation of intraocular tissue by reducing the force required to separate the portion of tissue from the intraocular matrix. In an example, the force required to perforate intraocular tissue can be reduced by shearing the intraocular tissue matrix with the slicing cut induced by rotary motion and separating the intraocular tissue matrix with the plunging cut.

Application of the force can include adjusting the pressure in the bore, such as increasing or decreasing pressure in the instrument bore including pressure near the distal cannula end 120B. By locating the distal cannula end 120B in proximity to intraocular tissue and decreasing pressure in the cannula bore near the distal cannula end 120B, such as with the pressure source 140 in communication with the instrument bore, intraocular tissue can be drawn into the cannula bore and against the cutting edge 122 of the distal cannula end 120B. The negative pressure level in the cannula bore can be adjusted, such as to adjust the magnitude of the perpendicular force generated by the distal cannula end 120B against the intraocular tissue, to control the plunge cut. Controlling the plunge cut can include controlling the rate of the plunge cut, such as the rate of advance of the distal cannula end 120B through the intraocular tissue.

In an example, the rate of the plunge cut can be increased, such as by adjusting the negative pressure in the cannula bore from a first negative pressure to a second negative pressure, such as where the absolute value of the first negative pressure is less than the absolute value of the second negative pressure. In an example the rate of the plunge cut can be decreased, such as by adjusting the negative pressure in the cannula bore from a third negative pressure to a fourth negative pressure, such as where the absolute value of the third negative pressure is greater than the absolute value of the fourth negative pressure.

Application of the force can include controlling the penetration depth of the distal cannula end 120B into the intraocular tissue. Drawing intraocular tissue into the cannula bore, such as by adjusting the level of negative pressure in the cannula bore, can create a seal between the distal cannula end 120B and the intraocular tissue, such as at least one of trabecular meshwork (TM) or the Schlemm's canal inner wall (SCIW) tissue. In an example, penetration of the distal cannula end 120B into 20 Schlemm's canal can interrupt the seal, such as to stop the advance of the distal cannula end 120B through the TM/SCIW tissue to control the penetration depth of the distal cannula end 120B into the intraocular tissue.

Application of the force can include capturing the perforated intraocular tissue, such as the tissue plug, for removal from the intraocular space. The tissue plug can be captured in the instrument bore, a portion of the cannula bore in proximity to the distal cannula end 120B.

Capturing the tissue plug can include perforating the intraocular tissue, such as the TM, with an exterior bevel cutting edge 122A, such as the tissue plug can be approximately equal to or less than the diameter of the instrument bore, such as the tissue plug can slide freely with respect to the interior cannula surface 134. Removing the tissue plug from the eye can include drawing the captured tissue plug through the instrument cannula, such as at least one of the cannula bore or the handle bore, with a differential pressure, such as a negative pressure level generated by a pressure source 140 in fluidic communication with the instrument bore.

Capturing the tissue plug can include perforating the intraocular tissue, such as the TM, with an interior bevel cutting edge 122B, such that the tissue plug can be approximately equal to or greater than the diameter of the instrument bore, such as the tissue plug can interfere with the interior cannula surface 134. Removing the tissue plug from the eye can include withdrawing the instrument 100 from the intraocular space and expelling the tissue plug from the instrument bore, such as with a differential pressure, such as a positive pressure level generated by the pressure source 140 in fluidic communication with the instrument bore.

At 606, the penetration depth of the distal cannula end 120B into the ocular tissue can be indicated or controlled. Indicating the penetration depth of the distal cannula end 120B can include sensing the penetration depth, such as by observing an indicia mark on the instrument 100. In an example, force can be applied to the instrument 100, such as to penetrate the intraocular tissue, and the indicia mark observed, such as with at least one of a gonioscope or an operative microscope, to indicate the penetration depth of the distal cannula end 120B into the intraocular tissue.

Indicating the penetration depth of the distal cannula end 120B can include sensing the penetration depth, such as with a sensor 150. In an example, sensing the penetration depth can include observing the indicia mark with a sensor 150, such as a visualization sensor. In an example, sensing the penetration depth can include sensing a distance between the intraocular tissue and a distance sensor, such as an ultrasonic distance sensor located at a fixed distance from the distal cannula end 120B.

Controlling the penetration depth of the distal cannula end 120B can include contacting the intraocular tissue with at least a portion of the instrument 100, such as the backstop 130. In an example, the backstop 130 can be located at a known distance from the distal cannula end 120B. Force can be applied to the instrument 100 to penetrate the intraocular tissue with the distal cannula end 120B and advanced into the intraocular tissue, such as until the backstop 130 contacts the intraocular tissue. Locating the backstop 130 on the distal cannula end 120B can include moving the backstop 130 with respect to the distal cannula end 120B, such as to control penetration depth of the distal cannula end 120B based on the individual anatomy of the patient, such as the measured TM thickness of the patient.

The distal cannula end 120B can include a backstop 130, such as a backstop 130 located at a specified distance from the distal cannula end 120B, configured to interfere with intraocular tissue, such as to prevent further penetration of the distal cannula end 120B into the intraocular tissue once the backstop 130 contacts the intraocular tissue. The specified distance can include at least one of a target penetration depth, such as desired depth of penetration into the intraocular tissue, or a safety threshold, such as a penetration depth beyond which ocular damage can occur. In an example, a target penetration depth can include a thickness of the intraocular tissue, such as the thickness of the trabecular meshwork of a patient to be treated with the instrument 100. In an example a safety threshold can include a percentage multiple of an intraocular tissue thickness, such as an average value of intraocular tissue thickness for a particular patient population. For example, where the thickness of trabecular meshwork/Schlemm's canal inner wall complex can range between about 50 micrometers and about 150 micrometers for a patient population, such as about 50-75 micrometers in the anterior TM region and about 100-130 micrometers in the posterior TM region, the safety threshold for the backstop 130 can include at least one of about 50%, 60%, 70%, 80%, 90%, or about 100% of the TM range for the patient population. In light of the above, it should be appreciated that other distances and dimensions are contemplated herein. In an example embodiment, backstop 130 has a maximum backstop distance of approximately 300 to 350 micrometers. In another example embodiment, backstop 130 has a maximum backstop distance of approximately 250 to 300 micrometers. An example embodiment may also include a distal cannula end 120B with a diameter of approximately 300 to 350 micrometers.

An indication of penetration depth can include an indication of linear force, such as an increase in the rate of linear force applied to the instrument 100 to advance the instrument 100 into the intraocular tissue. The increase in the rate of applied linear force, such as due to an increase in an indication of linear resistance, can indicate that the backstop 130 has contacted an intraocular tissue, such as the surface of the intraocular tissue.

An indication of penetration depth can include an indication of rotary force, such as an increase in the rate of rotary force applied to the instrument 100 to advance the instrument 100 into the intraocular tissue. The increase in the rate of rotary force, such as due to an increase in an indication of rotational resistance can indicate that the backstop 130 has contacted an intraocular tissue, such as the surface of the intraocular tissue.

An indication of penetration depth can include a visual indication, such as a direct image through a gonioprism and microscope. A sensor 150, such as a visualization sensor, can be directed toward the site of the penetration of the instrument 100 into the intraocular tissue. A user of the instrument 100 can observe the video image of the surgical field to monitor an indication of penetration depth, such as at least one of a scale applied to the exterior cannula surface 132 or contact of the backstop 130 with the intraocular tissue.

An indication of penetration depth can include an indication of position, such as the relative position of the instrument 100 with respect to the intraocular tissue. A sensor 150, such as a position sensor, can be attached to the instrument 100, such as located on the instrument 100 at a known distance from the distal cannula end 120B. In an example, the position sensor can be located at the position of the backstop 130. A user of the instrument 100 can monitor an indication of penetration depth from the position sensor, such as to control the force applied to the instrument 100 to advance the instrument 100.

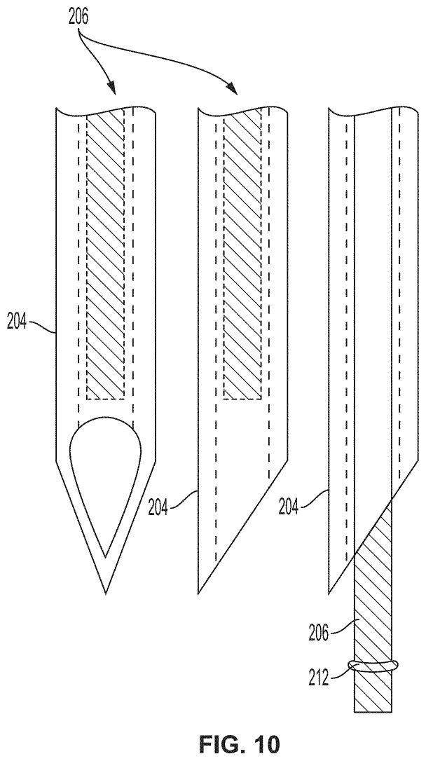

In an example embodiment, dissection trephine may be disposed with a hypodermic needle housing. For example, with reference to FIG. 7, instrument 200 may include a handle 202 and a hypodermic needle 204 disposed at the distal end of the handle 202. Instrument 200 further includes a dissection trephine 206, disposed within hypodermic needle 204. In a particular example, hypodermic needle 204 is one of a 27-gauge needle and a 25-gauge needle.

Handle 202 may further include a sliding button, which may be coupled to a proximal end of dissection trephine 206. FIG. 8 illustrates a cross-sectional view including this coupling between a sliding button 208 and the proximal end of dissection trephine 206. Sliding button 208 is disposed on an exterior of handle 202 and communicates with dissection trephine 206 via slot 210. For example, sliding button 208 is configured for manual sliding along slot 210, between a proximal location and a distal location. When sliding button 208 is disposed in the proximal location, dissection trephine 206 remains sheathed within hypodermic needle 204. When sliding button 208 is moved forward, and disposed in the distal location dissection trephine 206 is exposed at the distal end of hypodermic needle 204.

Specifically, sliding button 208 is moved forward along slot 210, as illustrated by FIG. 9. In an embodiment, sliding button 208 includes one or more ridges 212 for improved grip. FIG. 10 illustrates the dissection trephine 206 as it is exposed at the distal end of hypodermic needle 204 once sliding button is moved forward. The outer diameter of the dissection trephine 206 and the inner diameter of the hypodermic needle 204 are configured to ensure that the dissection trephine 206 may slide within hypodermic needle 204, while simultaneously ensuring a snug fit to prevent fluid from leaking into the hypodermic needle 204.

Generally, by disposing dissection trephine 206 within a hypodermic needle 204, the total number of required incisions can be reduced, allowing for more efficient in-office procedures. For example, dissection trephine 206 may be advantageously implemented without requiring an incision or any viscoelastic injection; rather, hypodermic needle 204 may be used for direct access to the anterior chamber via penetration.

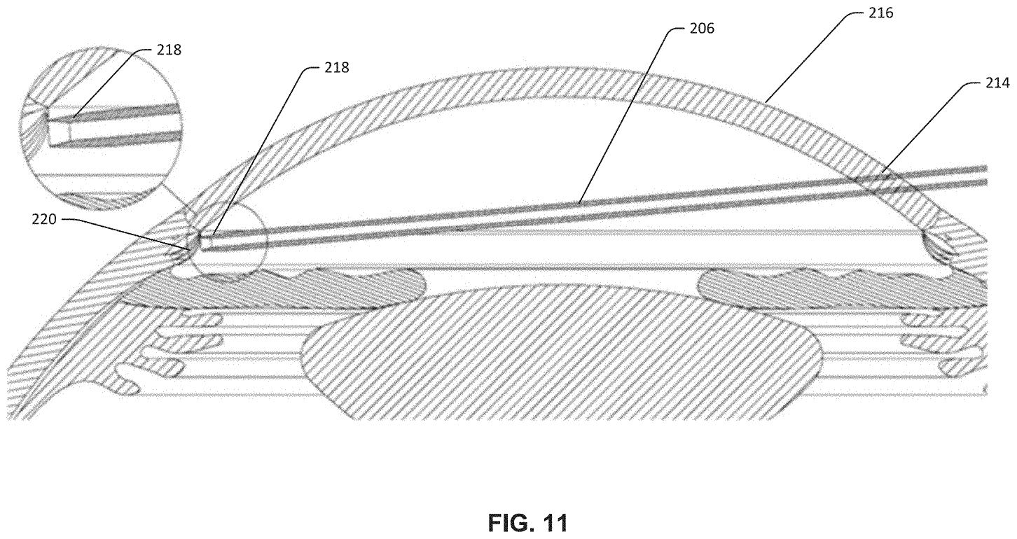

FIG. 11 illustrates a trephine, such as dissection trephine 206, inserted into the ocular environment. While FIG. 11 is illustrated with respect to dissection trephine 206, it should be appreciated that other trephines (e.g., dissection trephine 120 discussed above) are configured to be inserted in similar ways.

Namely, FIG. 11 illustrates a section view of an eye. Dissection trephine 206 is configured for insertion into the anterior chamber of the patient eye, such as through an incision 214 in the cornea 216 of the eye. Once inserted, the distal end 218 of dissection trephine 206 can be located proximally to intraocular tissue, such as to contact the intraocular tissue. In an embodiment, the dissection trephine 206 contacts trabecular meshwork 220. The distal end 218 of dissection trephine 206 may include a cutting surface, such as to cut, core or incise the intraocular tissue. In an example, the cutting surface can include a cutting trephine.

In a related embodiment, dissection trephine 206 and hypodermic needle 204 are collectively inserted through the cornea 216; once the hypodermic needle 204 has entered the anterior chamber of the eye, the dissection trephine 206 can readily be exposed at the distal end of the hypodermic needle 204 (as discussed previously).

The sleek profile disclosed herein provides for simple insertion, through the corneal incision 214, while simultaneously providing for maximum visibility during placement (e.g., into the trabecular meshwork 220). Handle 202 further provides improved surgeon control of the dissection trephine 206.

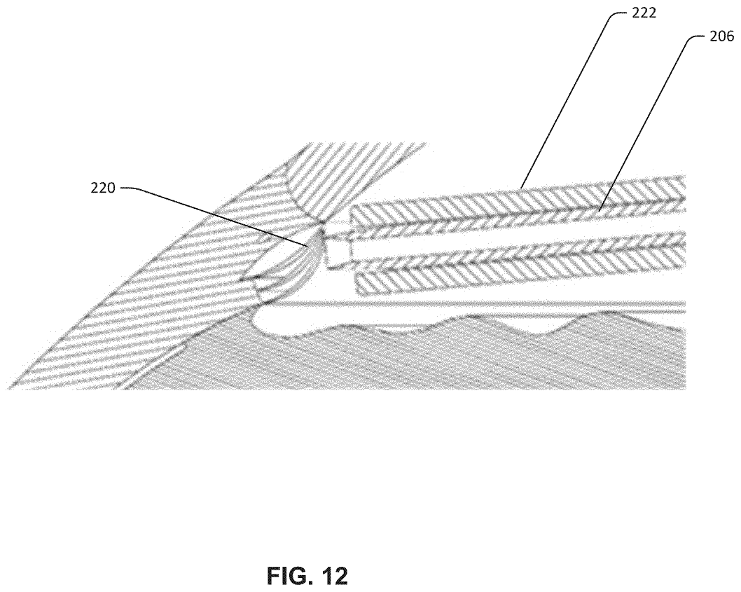

Similar to the functionality of the hypodermic needle 204 previously disclosed herein, in an embodiment, dissection trephine 206 may additionally or alternatively include a support collar 222, as illustrated by FIG. 12. Support collar 222 may be fixed partially or completely around the perimeter of dissection trephine 206. The proximal end of support collar 222 may advantageously act as a “limiter,” to prevent dissection trephine 206 from over insertion, which could result in undesirable puncturing of the sclera or other anatomical features.

When dissection trephine 206 is retracted, such that it is disposed within hypodermic needle 204 or support collar 222, the trephine 206 is protected during shipping and storage. This may additional provide sharps-protection from the distal end of trephine 206, thus improving safety. A retracted trephine 206 may further provide for safer entry through the corneal incision 214.

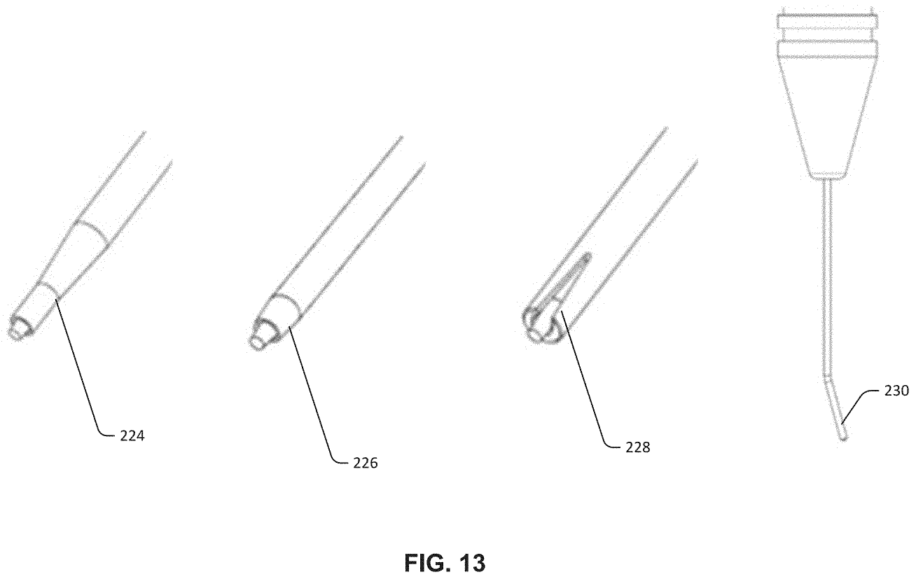

While support collar 222 is generally illustrated to have a flat-end in FIG. 12, it should be appreciated that other geometries are contemplated herein. Namely, for example, the distal end of support collar 222 could be multi-diameter 224 (e.g., “necked-down), chamfered 226, notched 228, or bent 230 as illustrated by FIG. 13. It should be appreciated that other related geometries are, likewise, contemplated herein.

For example, the trephine 206 may include a beveled edge at its distal end. In one embodiment, the beveled edge is a bevel-in edge, such that the outer diameter of the trephine 206 decreases in a distal direction toward the distal end; in this embodiment, the inner diameter of the trephine 206 is fixed. In another embodiment, the beveled edge is a bevel-out edge, such that the inner diameter of the trephine 206 increases in a distal direction toward the distal end; in this embodiment, the outer diameter of the trephine 206 is fixed. In another embodiment, the beveled edge is a bevel in-out edge, such that the inner diameter of the trephine 206 increases in a distal direction toward the distal end, while the outer diameter of the trephine 206 decreases in a distal direction toward the distal end. In an embodiment, the distal end of the trephine 206 includes one or more of a notch and a serrated edge, to ensure secure tissue engagement.

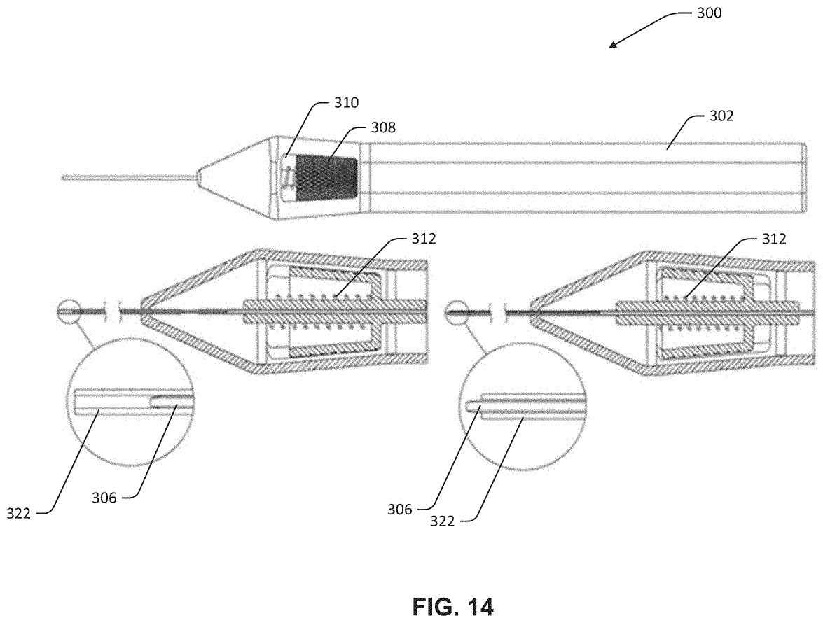

FIG. 14 illustrates an alternative instrument 300. Namely, whereas dissection trephine 206 of insertion device 200 was advanceable via sliding button 208, dissection trephine 306 of insertion device 300 is advanceable via a control wheel 308. Specifically, instrument 300 includes a handle 302, a support collar 322, and the dissection trephine 306 disposed within support collar 322. A proximal end of dissection trephine 306 may be coupled to control wheel 308.

In addition to illustrating alternative instrument 300 with control wheel 308 generally, FIG. 14 illustrates this alternative instrument 300 in both a non-activated state and an activated state. Specifically, for example, control wheel 308 may be initially disposed in a distal direction within slot 310. Instrument 300 may further include a spring 312 configured to bias control wheel 308 in the distal direction within slot 310; thus, spring 312 holds control wheel 308 and dissection trephine 306 in a retracted position.

Upon rotation of control wheel 308 by a surgeon, both control wheel 308 and dissection trephine 306 are advanced forward, such that dissection trephine 306 is advanced beyond the distal end of support collar 322.

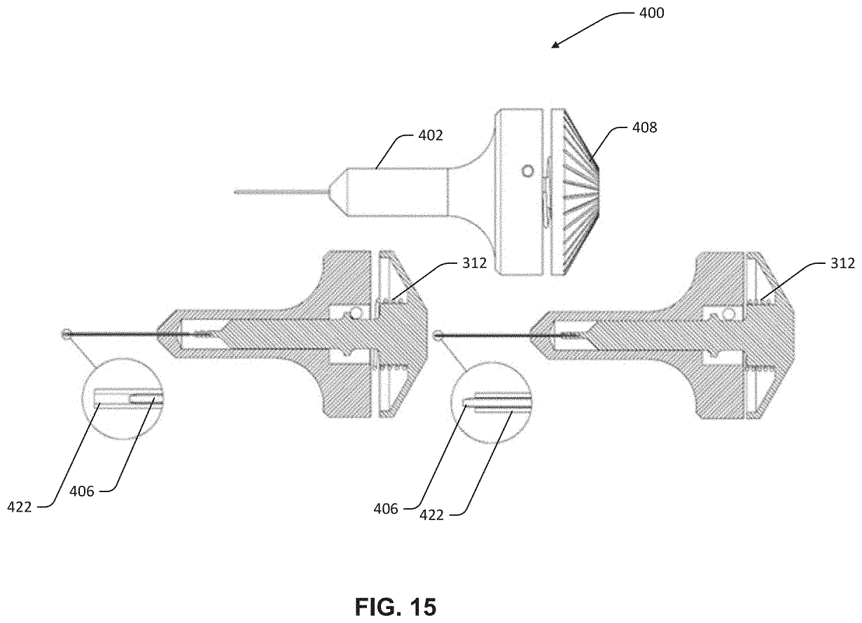

FIG. 15 illustrates another alternative instrument 400. Similar to instrument 300, dissection trephine 406 of instrument 400 is advanceable via rotation. Specifically, instrument 400 includes a handle 402, a support collar 422, and the dissection trephine 406 disposed within support collar 422. A proximal end of dissection trephine 406 may be coupled to thumb wheel 408.

In addition to illustrating alternative instrument 400 with thumb wheel 408 generally, FIG. 15 illustrates this alternative instrument 400 in both a non-activated state and an activated state. Specifically, for example, thumb wheel 408 may be initially disposed in a distal direction. Instrument 400 may further include a spring 412 configured to bias thumb wheel 408 in the distal direction; thus, spring 412 holds thumb wheel 408 and dissection trephine 406 in a retracted position.

Upon rotation of thumb wheel 408 by a surgeon, both thumb wheel 408 and dissection trephine 406 are advanced forward, such that dissection trephine 406 is advanced beyond the distal end of support collar 422.

Support tube 322 and support tube 422 provides stabilization and securement to ocular tissue during procedure, which can be especially helpful during trephine rotation (via control wheel 308 or thumb wheel 408). Furthermore, each of control wheel 308 and thumb wheel 408 may be configured to rotate a fixed amount, thus preventing dissection trephine from over-insertion and thus avoiding excessive penetration and/or cutting. Rotatable trephines, such as trephine 306 and trephine 406 may improve coring operations, requiring less axial force for penetration and/or cutting.

Although the internal mechanisms and ergonomics of instruments 100, 200, 300, and 400 are varied, it should be appreciated that any aspects or features of any individual instrument is, likewise, applicable to any other individual instrument.

The above description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The invention is claimed as follows:

1. An instrument to perforate ocular tissue, the instrument comprising:
a handle configured to be grasped by a user, the handle including a proximal handle end and a distal handle end; and
a dissection cannula configured for at least partial insertion into a patient eye, the dissection cannula including:
a proximal shaft end configured to interface with the handle;
a distal cannula end including an opening into a cannula bore; and
an indicator spaced from the distal cannula end at a spacing to indicate or control insertion depth of the distal cannula end into the eye.

2. The instrument of claim 1, wherein the distal cannula end includes a beveled edge or trephine to penetrate ocular tissue.

3. The instrument of claim 2, wherein the beveled edge includes at least one of a single bevel or a double bevel.

4. The instrument of claim 3, wherein the beveled edge is sloped toward an interior surface of the cannula.

5. The instrument of claim 1, comprising a serrated edge extending about at least a portion of the distal cannula end.

6. The instrument of claim 5, wherein the serrated edge extends about the entire distal cannula end.

7. The instrument of claim 1, wherein the indicator includes a backstop extending laterally wider than the distal cannula end.

8. The instrument of claim 7, wherein the backstop includes a collar extending about at least a portion of the exterior surface of the cannula.

9. The instrument of claim 8, wherein the indicator includes an elastic band configured to be user-adjustable in location on the cannula to define the spacing.

10. The instrument of claim 1, comprising a pressure source in communication with the cannula bore to adjust pressure in the cannula bore.

11. The instrument of claim 10, wherein the pressure source is configured to generate enough negative pressure in the cannula bore to draw ocular tissue into the bore.

12. The instrument of claim 11, wherein the pressure source is configured to generate enough negative pressure in the cannula bore to cut the ocular tissue against the distal cannula end.

13. The instrument of claim 1, wherein the handle is integral with the dissection cannula.

14. A method of using an instrument, the method comprising:
inserting a distal cannula end of the instrument into a patient eye to locate the distal cannula end against intraocular tissue; and
applying force between the instrument and the eye to perforate the intraocular tissue and capture the perforated intraocular tissue for removal from the eye.

15. The method of claim 14, wherein inserting includes locating the instrument against a trabecular meshwork of the eye.

16. The method of claim 15, wherein applying force includes applying at least one of a linear force or a rotary force to the instrument located against the ocular tissue to perforate the tissue.

17. The method of claim 16, wherein applying force includes applying linear force.

18. The method of claim 16, wherein applying force includes applying rotary force.

19. The method of claim 14, wherein applying force includes generating negative pressure in a bore of the instrument.

20. The method of claim 19, comprising indicating or controlling penetration depth of the distal cannula end into the ocular tissue.
